(19) United States
(12) Patent Application Publication
(10) Pub. No.: US 2002/0080329 A1
(43) Pub. Date: Jun. 27, 2002

(54) Corneal endothelium analysis system

(76) Inventor: Tatsuya Kasahara, Nishinomiya-shi (JP)

Correspondence Address: MARSHALL, GERSTEIN & BORUN, 6300 SEARS TOWER, 233 SOUTH WACKER, CHICAGO, IL, 60606-6357, US

(21) Appl. No.: 09/993,971

(22) Filed: Nov. 14, 2001

(30) Foreign Application Priority Data
Dec. 27, 2000 (JP) 2000-397149

(51) Int. Cl. A61B 3/00

(52) U.S. Cl. 351/200

(57) ABSTRACT

A corneal endothelium analysis system comprises: a terminal; and a server connected to the terminal through a network. The terminal comprises means for photographing corneal endothelium and first transmission/receiving means for transmitting an image of the photographed corneal endothelium to the server and receiving an analysis result from the server, and the server comprises second transmission/receiving means for receiving the image of the corneal endothelium from the terminal and transmitting the analysis result to the terminal, and means for analyzing the image of the corneal endothelium.

### BACKGROUND OF THE INVENTION

1. Field of the Invention

The present invention relates to a corneal cell analysis system. More particularly, the present invention relates to a corneal cell analysis system that photographs corneal of a subject by himself/herself, and calculates and provides numeric values used for evaluating corneal cells based on an image of the photographed corneal of the subject.

2. Description of the Related Art

Conventionally, a device for observing and photographing corneal endothelium and a device for measuring corneal thickness are known. In this device, a subject looks into an objective of the device in accordance with instructions of a tester and fixes his/her eyes on a fixation target, and when judging that the subject is ready for photographing, the tester photographs the corneal of the subject. The tester uses an analyzer to perform image processing of the corneal image and obtains numeric values for a cell density (the number of cells per unit area), an average area of cells, standard deviation of cell areas, and the like. Based on these values, the tester evaluates the condition of the corneal of the subject.

With the conventional device, the tester needs to give many instructions to the subject during photographing. In addition, in analysis of the obtained corneal image, the tester must conduct various operations such as judgment of the image. In this case, there is a possibility that the judgment and operation of the tester entail errors. Besides, it is necessary to always install the photographing device and the analyzer close to each other. Under such circumstances, it is difficult to test many users and the system cost of installation is increased.

### SUMMARY OF THE INVENTION

The present invention has been developed for obviating the above-described problem and an object of the present invention is to provide a corneal endothelium analysis system capable of providing accurately analyzed data of the corneal endothelium with ease and at a low cost according to the frequency of use.

According to the present invention, there is provided a corneal endothelium analysis system comprising: a terminal; and a server connected to the terminal through a network, wherein the terminal comprises means for photographing the corneal endothelium and first transmission/receiving means for transmitting an image of the photographed corneal endothelium to the server and receiving an analysis result from the server, and the server comprises second transmission/receiving means for receiving the image of the corneal endothelium from the terminal and transmitting the analysis result to the terminal, and means for analyzing the image of the corneal endothelium.

With the analysis system, it is not necessary to place the means for photographing the corneal endothelium and the means for analyzing the corneal endothelium adjacent to each other. Also, it is possible to connect a plurality of (a number of) terminals to one (a few number of) servers through the network. Consequently, the terminal and the server can be respectively installed with a much higher degree of freedom, and can be employed in a variety of configurations. In addition, a terminal having a photographing function can be configured at a low cost. Further, the subject can be photographed without the tester.

It is preferable that the photographing means comprises an anterior segment observing optical system, an alignment optical system, an illumination optical system, a photographing optical system, and a photographing control portion, for automatically photographing the corneal endothelium, because the subject can easily photograph his/her eyes for himself/herself.

It is preferable that the terminal further comprises first storing means for storing the image of the corneal endothelium, first control means for controlling the first storing means and the first transmission/receiving means, and means for operating the first control means, because the terminal can be connected to the server where the image of corneal endothelium photographed by the subject is analyzed.

It is preferable that the terminal further comprises means for displaying the image of the corneal endothelium and the analysis result under control of the first control means, because the user at the terminal including the subject can immediately know the analysis result of the corneal endothelium.

It is preferable that the terminal further comprises means for outputting the image of the corneal endothelium and the analysis result under control of the first control means, because the user who used the terminal can preserve the image of the corneal endothelium and the analysis result as necessary or perform the other various operations.

It is preferable that the terminal further comprises means for entering subject information and/or information used for obtaining an access right of the server under control of the first control means, because unrestricted use of the system by a third (disinterested) person is prohibited and the subject information can be managed. The subject information may include a subject name, a birthday of the subject, an illness history of the subject, or the like.

It is preferable that the terminal further comprises second storing means for storing control information enabling the photographing means to perform photographing on condition that permission to use the photographing means is obtained from the server, because unrestricted use of the photographing means is prevented and the automatic photographing becomes possible after the use is permitted.

It is preferable that the server further comprises second control means and third storing means, the second control means is adapted to control the third storing means, the second transmission/receiving means, and the analyzing means, and the third storing means is adapted to store at least one analysis result selected from a group comprising a minimum cell area of the corneal endothelium, a maximum cell area of the corneal endothelium, an average cell area of the corneal endothelium, the number of analyzed corneal endothelium cells, the number of corneal endothelium cells per unit area, standard deviation of corneal endothelium cell areas, a variation coefficient of the corneal endothelium cell areas and a rate of appearance of a hexagonal cell, and the image of the corneal endothelium. In this system, the second control means (e.g., computer) serves to control the analysis of the corneal endothelium image, storage of the analysts result, and transmission of the analysis result. Accordingly, the subject can easily refer to and utilize his/her own past data at any terminal of this system, i.e., at another hospital or shop where terminals are located.

It is preferable that the terminal further comprises means for observing the condition of a subject, and is adapted to transmit information of the condition of the subject observed by the observing means to the server, and the server comprises sixth storing means for storing information with which it is judged whether or not the subject can be photographed depending on the condition of the subject. This is because the photographing can be started only when the condition of the subject to be photographed by the photographing means, for example, the position of the face of the subject facing the terminal, the position of the eye to be tested, and the direction toward which the eyes are fixed are appropriate.

It is preferable that the server is adapted to compare received information of the condition of the subject to the information stored in the sixth storing means and transmit an instruction pertaining to the subject condition to the terminal, and the terminal comprises means for giving the instruction received from the server, because the subject can be in the photographed state in accordance with the instructions from the server.

It is preferable that the server further comprises fourth storing means for storing control information used for accounting on the terminal for the use of the photographing means and/or fifth storing means for storing control information used for accounting on the terminal for transmission of the analysis result to the terminal, because the system is suitable as commercially available means.

It is preferable that the server further comprises seventh storing means for storing control information used for transmitting information pertaining to expendable part replacement timing to the terminal according to how the terminal is used, because the maintenance of the whole system can be managed at the server end even in the system including at least one remote terminal. So, in the system in which the photographing means comprises a photographing light source and a detection light source as the expendable parts, the maintenance is almost manageable at the server end, because these expendable parts are main expendable parts. The “photographing light source” includes a xenon tube or the like as an illumination light source that emits light in photographing as mentioned later, and the “detection light source” includes a light source of the alignment light, a focusing lamp, or the like as mentioned later.

The above and further objects and features of the invention will more fully be apparent from the following detailed description with accompanying drawings.

### BRIEF DESCRIPTION OF THE DRAWINGS

FIG. 1 is a block diagram showing an embodiment of a corneal endothelium analysis system of the present invention;

FIG. 2 is a block diagram showing an example of a user terminal in the corneal endothelium analysis system of FIG. 1;

FIG. 3 is a schematic view showing an example of a photographing device in the user terminal of FIG. 2;

FIG. 4 is a block diagram showing an example of a server in the corneal endothelium analysis system of FIG. 1;

FIG. 5 is a flowchart showing a procedure for analyzing corneal endothelium in the system of FIG. 1 based on communication between the user terminal and the server;

FIG. 6 is a flowchart showing a procedure for analyzing corneal endothelium in the system of FIG. 1 based on communication between the user terminal and the server;

FIG. 7 is a flowchart showing a procedure for analyzing corneal endothelium in the system of FIG. 1 based on communication between the user terminal and the server;

FIG. 8 is a flowchart showing a procedure for analyzing corneal endothelium in the system of FIG. 1 based on communication between the user terminal and the server;

FIG. 9 is a front view showing an example of a monitor screen in the user terminal in the corneal endothelium analysis system of FIG. 1;

FIG. 10 is a front view showing another example of the monitor screen in the user terminal in the corneal endothelium analysis system of FIG. 1;

FIG. 11 is a front view showing still another example of the monitor screen in the user terminal in the corneal endothelium analysis system of FIG. 1;

FIG. 12 is a front view showing a further example of the monitor screen in the user terminal in the corneal endothelium analysis system of FIG. 1; and

FIG. 13 is a front view showing a still further example of the monitor screen in the user terminal in the corneal endothelium analysis system of FIG. 1.

### DETAILED DESCRIPTION OF THE PREFERRED EMBODIMENT

Now, a preferred embodiment of a corneal endothelium analysis system according to the present invention will be described with reference to accompanying drawings.

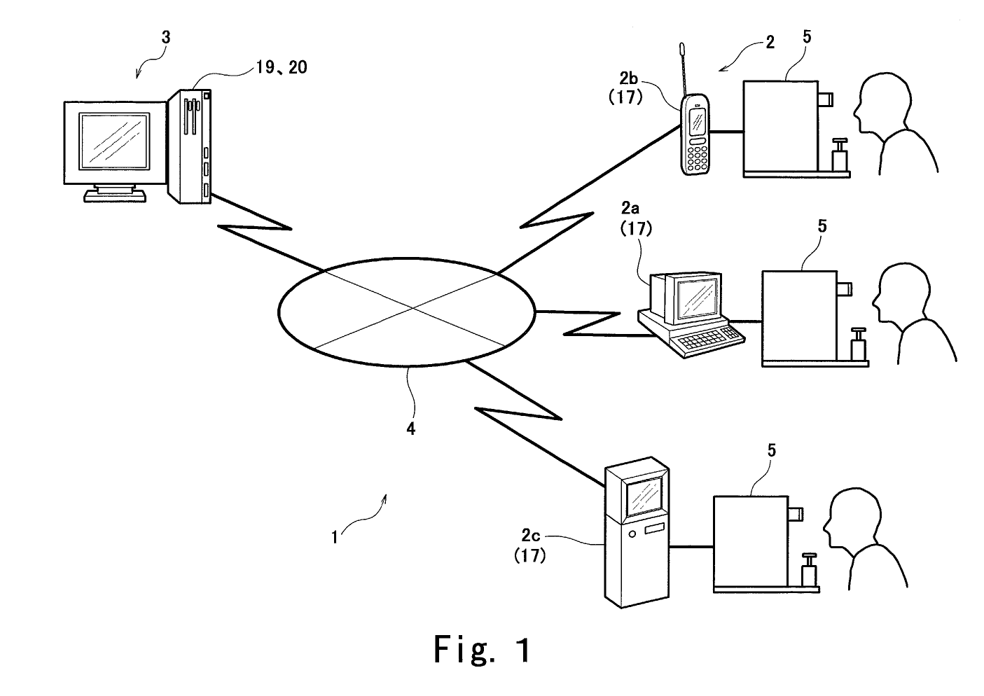

FIG. 1 is a block diagram showing an embodiment of the corneal endothelium analysis system (hereinafter simply referred to as a system) of the present invention.

Referring now to FIG. 1, a system 1 comprises a user terminal 2 and a server machine 3 in a center which are connected through a network 4. The user transmits a corneal endothelium image to be analyzed from the user terminal 2 to the server machine 3. The server machine 3 analyzes the image under predetermined conditions and transmits the analysis result as a reply to the user terminal 2. The “user” is not limited to a subject who goes through photographing or analysis but may be a proprietor of the user terminal 2 or a person who occupies the terminal 2 during a given period by a contract. Therefore, hereinbelow, the user and the subject are called “user or the like”.

The user terminal 2 is capable of performing communication of information of an image or the like, and comprises a personal computer 2a, a cellular phone 2b, an interactive kiosk 2c, and a personal digital assignment (PDA) that is not shown. The user terminal 2 is provided with a photographing device 5 for photographing corneal endothelium of an eye E to be tested (hereinafter “eye E”) of the subject and first transmission/receiving means 17 for transmitting the obtained image information to the server machine 3 and receiving the analysis result of the image from the server machine 3.

The server machine 3 is provided with analyzing means 20 for analyzing the image information and second transmission/receiving means 19 for receiving the image information and transmitting the analysis result to the user terminal 2. The transmission/receiving means 17, 19 are interfaces to enable communication through various networks and are also referred to as a network control portion.

In this embodiment, the network 4 is capable of performing communication of information such as the image information or the like according to a configuration of the user terminal 2. The network 4 is the combination of plural types of networks when the user terminal 2 is configured in various ways. Specifically, in case of the personal computer of FIG. 1, the network 4 is generally Internet/Intranet. In case of the cellular phone, the network 4 is generally a web data communication network for the cellular phone managed by a cellular phone communication agency. The “cellular phone” refers to a cellular phone in a broad sense and includes a PHS (Personal Handyphone System) and the like advantageous in data communication, or otherwise these may be replaced by the PDA. Further, these days, in case of numerous interactive kiosks located at stores or the like, the network 4 is a dedicated network utilizing satellite communication managed by contact lens shops at which the interactive kiosks are located.

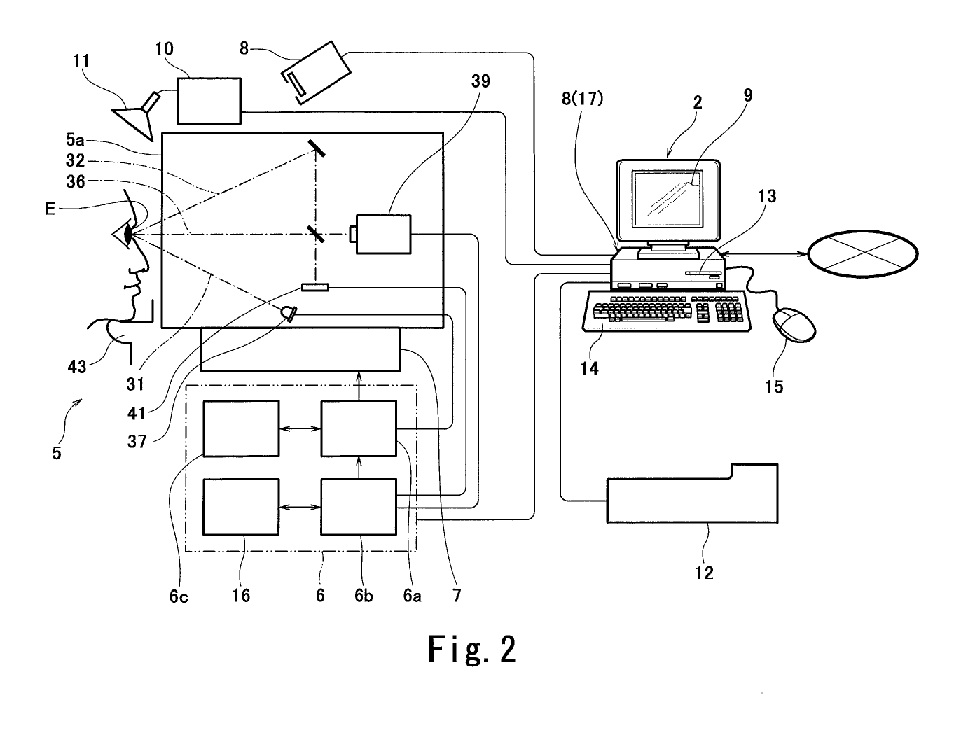

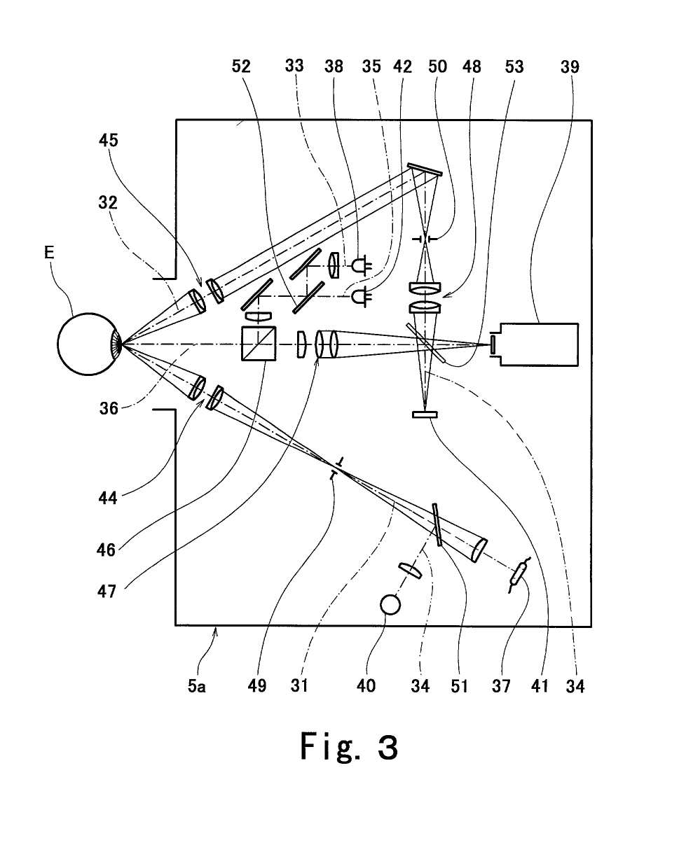

As shown in FIG. 2, the photographing device 5 provided in the user terminal 2 comprises a photographing system 5a for photographing the corneal endothelium of the eye E and a control portion 6 for controlling activation of the photographing system 5a to enable the system 5a to photograph the cells of the eye E. The photographing system 5a is adapted to observe and photograph an anterior segment of the eye E, and hence, the corneal endothelium of the eye E. The control portion 6 comprises a photographing control portion 6a, an image input/output control portion 6b, image storing means 16 for storing the image, and control information storing means 6c for storing information pertaining to control after acquiring permission to use the photographing device 5. The image storing means 16 corresponds to first storing means and the control information storing means 6c corresponds to second storing means. The photographing control portion 6a controls a photographing system drive mechanism 7 to activate the alignment and focusing of the photographing system 5a with respect to the eye E by using the control information stored in the control information storing means 6c and instructs the photographing system 5a to conduct photographing when the condition suitable for photographing is satisfied. The image input/output control portion 6b controls the image storing means 16 to store the image of the anterior segment and the image of the corneal endothelium. Reference numeral 43 denotes a vertically movable chin table on which a face of the subject is put in a stationary state when the subject is looking into the objective 45 (see FIG. 3) to have the eye E photographed. The control portion 6 may be provided only in the server machine 3 without being provided in the terminal 2, thereby controlling the photographing device 5 of the terminal 2 through the network 4.

As shown in FIG. 3, the photographing system 5a of the photographing device 5 comprises an illumination optical system 31 for illuminating the anterior segment of the eye E obliquely from forward, a photographing optical system 32 for photographing light reflected on a surface of the anterior segment of the eye E, an alignment optical system 33 for irradiating the anterior segment of the eye E from front with alignment light for alignment of a photographing optical axis and photographing the light reflected on the corneal, a focusing optical system 34 for performing focusing of the photographing system 5a on the corneal endothelium as a portion to be photographed, and a target optical system 35 for making the subject fix his/her eyes on a fixation target lamp 42 to thereby fix the direction of the eye E. When alignment is performed by using the alignment optical system 33, it is necessary to continuously photograph the anterior segment of the eye E. For this reason, the anterior segment observing system 36 is integrated with the alignment optical system 33. In other words, optical axes of the systems 33, 36 extending toward the eye E correspond with each other.

The illumination optical system 31 includes a xenon tube 37 as an illumination light source. The alignment optical system 33 includes a light-emitting diode 38 as a light source of the alignment light and a television camera 39. The television camera 39 is used in the observation of the anterior segment performed by the anterior segment observing system 36 and is also used in the photographing of the corneal endothelium performed by the photographing optical system 32. The focusing optical system 34 includes a focusing lamp 40 and an image detecting sensor 41 such as a line sensor or an area sensor. The optical axis of the focusing optical system 34, reaching the eye E, substantially corresponds with the optical axis of the illumination optical system 31 and the following optical axis thereof from the eye E substantially corresponds with the optical axis of the photographing optical system 32. The alignment light and the focusing light are both near infrared light and the light of the fixation target lamp 42 and the illumination light for photographing are both visible light.

In FIG. 3, reference numeral 44 denotes an illumination lens, 45 denotes an objective, 46 denotes a beam splitter, 47 denotes an anterior segment photographing lens, 48 denotes an imaging lens, 49 denotes a slit, and 50 denotes a field diaphragm. Reference numerals 51, 52 denote half minors that reflect infrared light and transmit the visible light and reference numeral 53 denotes a half mirror that reflects visible light and transmits the infrared light.

In the alignment optical system 33, the near infrared light is emitted from the light-emitting diode 38 and the anterior segment of the eye E is irradiated with the near infrared light from its front. A luminescent spot (Purkinje image) corresponding to an image of this light reflected on the corneal of the eye E is sent to the television camera 39. Based on the Purkinje image, the photographing system 5a is moved in X and Y directions, i.e., in the direction perpendicular to an axis extending toward the apex of the corneal of the eye E to make the photographing optical axis correspond with the apex of the corneal. Specifically, the photographing control portion 6a detects the position of the Purkinje image in the anterior segment image in accordance with a signal from the image input/output control portion 6b and the photographing system drive mechanism 7 drives the photographing system 5a to be moved in accordance with a signal from the photographing control portion 6a to allow the Purkinje image to reach apex of the corneal. Thereby, alignment is performed. In general, focusing is performed simultaneously with the alignment.

In the focusing optical system 34, slit light for detecting focusing is emitted from the focusing lamp 40, travels along the optical axis of the illumination optical axis system 31, and reaches the eye E. The slit light is reflected on the anterior segment, travels through a focusing slit (not shown) along the optical axis of the photographing optical system 32, and reaches the image detecting sensor 41, to be received. When a point (focusing point) at which the optical axis of the illuminating optical system 31 and the optical axis of the photographing optical system 32 intersect is situated on a portion of the eye E to be photographed, the image detecting sensor 41 detects the reflected light of focusing detection light. At this time, the reflected light passes through the slit. This is because the focal point of the photographing optical system 32 is set at the focusing point. The optical axis of the alignment optical system 33 and the optical axis of the anterior segment observing system 36 are configured to pass through the focusing point. In accordance with the signal from the image input/output control portion 6b, the photographing control portion 6a controls the photographing system 5a to be moved so as to be close to or apart from the eye E (Z direction in FIG. 2), thereby aligning the focusing point with the portion of the eye E to be photographed. This alignment is called focusing.

After the alignment and focusing are performed, the photographing control portion 6a causes the xenon tube 37 to emit light, and the corneal endothelium is photographed.

Thus far, the configuration and function of the photographing device 5 of the present invention have been described.

In photographing of the corneal endothelium, it is necessary to appropriately position the subject with respect to the photographing device 5 prior to the alignment or the like controlled by the control portion 6. A CCD (charge coupled device) camera 8 as means for observing the condition of the subject is connected to the user terminal 2. The anterior segment observing system 36 in the photographing system 5a is also adapted to detect the position of the eye E of the subject, and is therefore considered as one of the subject observing means.

The user terminal 2 is provided with a monitor display 9 (hereinafter simply referred to as a monitor) as display means and instructing means and a voice generating circuit 10 and a speaker 11 as instructing means. The monitor 9 is capable of displaying the image of the anterior segment and the image of the corneal endothelium which are photographed by the photographing device 5 and an analysis result sent from the server machine 3 as mentioned later. Also, when the eye E is photographed by the photographing device 5, the instructions or advices to the subject issued by the server machine 3 are displayed on the monitor 9 or given in the form of voice through the speaker 11. Further, when the server machine 3 judges that the image sent to the server machine 3 does not satisfy certain requirements such as brightness and focused state of the image which are necessary for analysis, an instruction for re-photographing is given through the monitor 9 or the speaker 11, whereas when the server machine 3 judges that the image satisfies the requirements, a message stating “image is being analyzed” or the like is displayed on the monitor 9. Various types of screens on which information pertaining to the subject is input with a key board 14 and a mouse 15 mentioned later are displayed on the monitor 9. The information pertaining to the subject includes a subject name, birthday of the subject, illness history of the subject, and so on.

The terminal 2 is further provided with a printer 12 as output means, and an ID card reader 13, a keyboard 14, and a mouse 15 as the input meas. Automatically or by the operation of the key board 14 or the mouse 15 by the user or the like, the corneal endothelium image, the anterior segment image, and the analysis result are printed out by the printer 12.

The ID card reader 13 is adapted to read an identifier (ID) of the user or the like under contract from an ID card in which the ID is stored. The key board 14 and the mouse 15 are used to enter the subject information or the like as described above. The subject information and the ID may be transmitted together to the server machine 3, or transmitted independently at different timings. The user ID and the subject information are stored in a data base 22a for storing information of the user or the like which is provided in the server machine 3. The user ID is used to authenticate the user and the subject information is used to retrieve and confirm the subject. For the convenience for the subject, subject information data base for storing the subject information may be provided in the terminal 2. The image taken in the past or the analysis result obtained in the past may be stored in this data base as associated with the subject information.

Image storing means 16 is connected to the terminal 2. The image of the anterior segment and the image of the corneal endothelium of the eye E which are photographed by the photographing system 5a are stored in the image storing means 16. The stored image is, after completion of photographing operation, transmitted by the first transmission/receiving means 17 of the terminal 2 to the server machine 3, automatically or by the operation of the key board 14 or the mouse 15 performed by the subject. The other information transmitted from the first transmission/receiving means 17 to the server machine 3 may include the user ID, the subject information, and information indicating the condition of the subject (including anterior segment) which is obtained by the CCD camera 8 and the anterior segment observing system 36.

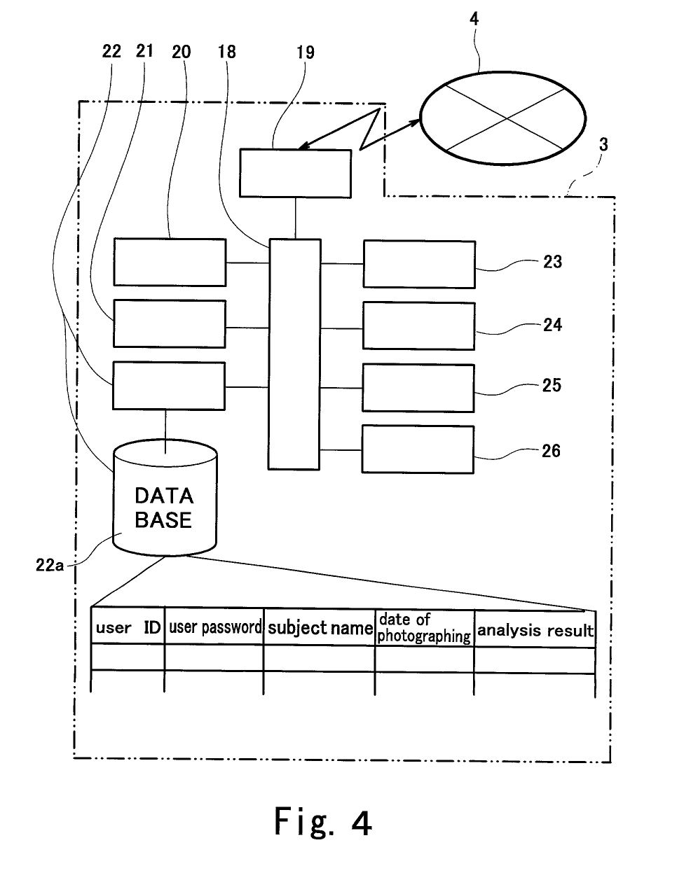

With reference to FIG. 4, the server machine 3 will be described. The server machine 3 is provided with a control portion 18 comprising CPU or the like, the second transmission/receiving means 19 for receiving the information pertaining to the anterior segment image and the corneal endothelium image which are transmitted from the first transmission/receiving means 17 of the terminal 2, and the analyzing means 20 for analyzing the received corneal endothelium image for various purposes. The analyzing means 20 comprises ROM or the like and contains a program for judging (preanalyzing) whether or not the received image satisfies analysis requirements prior to the analysis, and a program for analyzing an appropriate image in various ways. These programs are executed by the control portion 18.

Items to be analyzed include a minimum cell area of the corneal endothelium, a maximum cell area of the corneal endothelium, an average cell area of the corneal endothelium, the number of analyzed corneal endothelium cells, the number of corneal endothelium cells per unit area, standard deviation of the corneal endothelium cell areas, a variation coefficient of the corneal endothelium cell areas, the rate of appearance of a hexagonal cell, and so forth. To obtain such numeric values from the image information, image analysis such as smoothing, shading compensation, floating binarization, thinning, and dot noise reducing is carried out.

The server machine 3 is provided with analysis result storing means 21 comprising RAM or the like for storing the received corneal endothelium image and the analysis result obtained by the analyzing means 20. The analysis result storing means 21 corresponds to the third storing means. The analysis result stored in the analysis result storing means 21 is transmitted to the first transmission/receiving means 17 of the terminal 2 by the second transmission/receiving means 19.

The server machine 3 is further provided with authentication means comprising the data base 22a, first accounting information storing means 23, and second accounting information storing means 24. The user authentication means 22 is adapted to authenticate the user under contract and confirm the subject based on the received user ID and subject information, and the data base 22a. The first accounting information storing means 23 is adapted to store the user ID and information of accounting on the user for the use of the photographing device 5 by the user. The second accounting information storing means 24 is adapted to store the user ID and the information of accounting on the user for the transmission of the analysis result. Information pertaining to the account balance of the user is stored in the accounting information storing means 23, 24. For the authenticated user, it is judged whether or not the user is allowed to use the photographing device 5 and to be analyzed (or allowed to receive the analysis result), based on the balance checking by the accounting information storing means 23, 24. When “pay in advance” is desired and the balance is short of charged fee of the photographing device 5, the photographing device 5 is not activated, which is transmitted together with the necessary fee, and displayed on the monitor 9. When the balance is sufficient for the charged fee of the use of the photographing device 5 but is short of the analysis fee, this is displayed and short fee is charged by displaying it on the monitor 9.

When “pay later” is desired, fee is displayed on the monitor 9 after completion of the photographing (transmission of the analysis result) and charged according to a predetermined deal. Regardless of “pay in advance” or “pay later”, the user or the like can select cancellation of the photographing (analysis) before the start of the photographing (analysis). The first accounting information storing means 23 corresponds to fourth storing means and the second accounting information storing means 24 corresponds to fifth storing means.

Instruction and advice information storing means 25 is connected to the control portion 18. For various situations in which the subject or his/her anterior segment is difficult to photograph, instructions and advices for guiding the user into a photographing condition, are stored in the instruction and advice information storing means 25. The instruction and advice information storing means corresponds to sixth storing means. Comparison is made between the condition of the subject or the condition of the anterior segment which is transmitted from the terminal 2 and appropriate condition information or the like stored in the instruction and advice information storing means 25 and necessary instructions and advices are transmitted to the first transmission/receiving means 17 of the terminal 2 by the second transmission/receiving means 19. The analyzing means 20 judges from pre-analysis whether or not the received image satisfies conditions (the brightness, focusing, or the like) required for analysis. Then, as mentioned previously, the instruction for re-photographing or the massage stating “the image is being photographed” or the like is transmitted to terminal 2.

Further, maintenance information storing means 26 is connected to the control portion 18. Maintenance information such as replacement time of expendable parts of the terminal 2 is stored in the maintenance information storing means 26. The maintenance information storing means 26 corresponds to seventh storing means. Information to identify the parts of the terminal 2 requiring replacing in a short time, information of replacement periods of the parts, and information of replacement time of the expendable parts in each terminal and the like, are stored in the maintenance information storing means 26. Also, control information used for transmitting these information at an appropriate time to the first transmission/receiving means 17 of the terminal 2 by the second transmission/receiving means 19 to inform the terminal 2 of the expendable parts and the replacement time thereof is also stored in the storing means 26. The parts to be replaced may include the xenon tube 37, the light-emitting diode 38, the focusing lamp 40, the fixation target lamp 42, and the like.

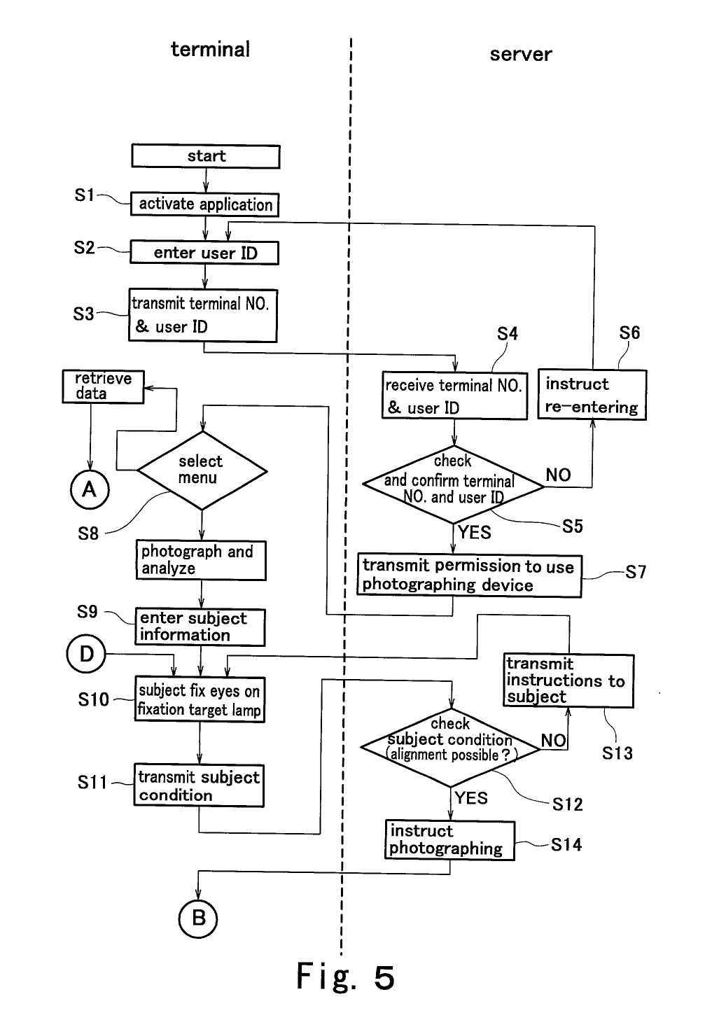

Subsequently, flow indicating how the server machine 3 analyzes the corneal endothelium image in the state in which the user terminal 2 is connected to the server machine 3, will be explained with reference to FIGS. 5 through 8.

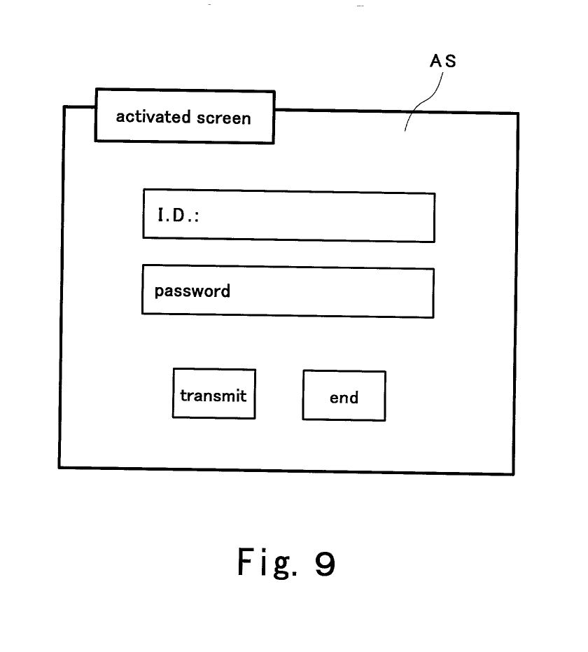

Referring to FIG. 4, first of all, an application of the user terminal 2 is activated to access to the server machine 3 from the user terminal 2 (Step 1 : hereinafter Step is represented by “S”). Thereby, an activated screen (AS) is displayed on the monitor 9 as shown in FIG. 9. The user or the like makes the ID card reader 13 read in the user ID from the ID card or enters the user ID on the activated screen AS using the key board 14 or the mouse 15 (S2). Then, the user or the like enters the pass word and transmits it to the server machine 3(S3). At this point, the user or the like may enter the subject information and transmit it to the server machine 3.

In the server machine 3, the user authentication means 22 checks the received (S4) identification number of the terminal 2 and the received user ID (and subject information) with the data base 22a for authentication (S5). In some cases, the balance of the user or the like is checked based on the first accounting storing means 23 and the second accounting storing means 24. If the user or the subject cannot be authenticated, a message stating “you are not authenticated and re-entering is required” is transmitted to the terminal 2 (S6). In the terminal 2, this message is displayed on the monitor 9. At this point, accounting may be performed when “pay later” is adopted.

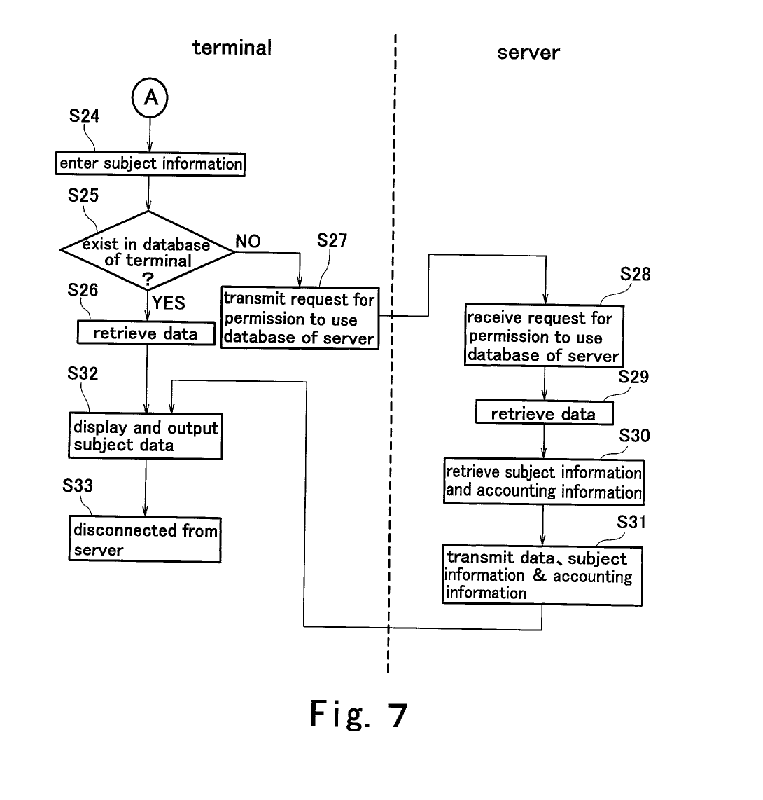

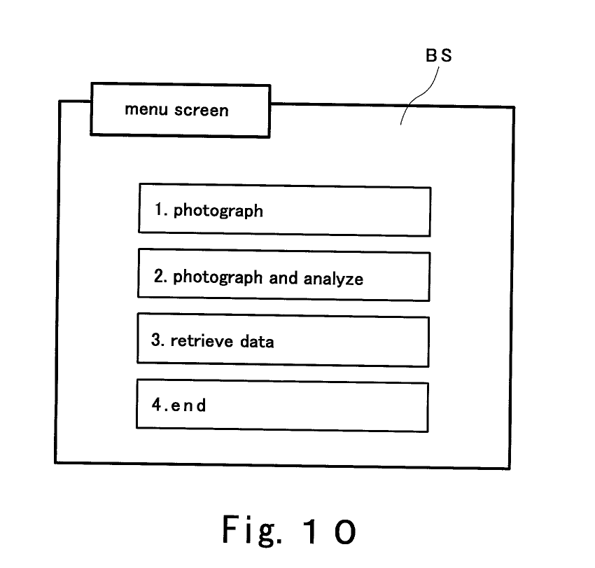

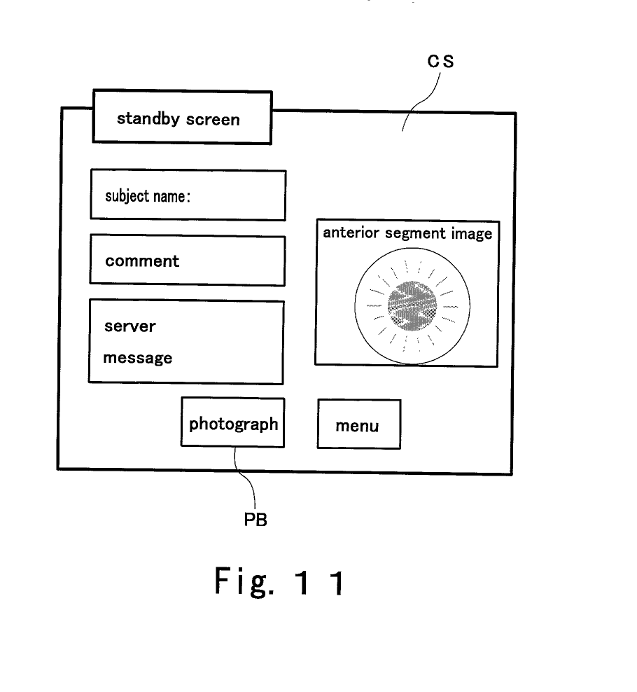

When authentication is complete, the server machine 3 transmits permission to use the photographing device 5 (S7). Then, a menu screen BS is displayed on the monitor 9 as shown in FIG. 10. The user or the like selects desired service among data retrieval, photographing, analysis displayed on the menu screen BS (S8). The “data retrieval” refers to extraction of illness history of the subject, a past cell image of the subject, the past analysis result of the subject, and the like. The “photographing” refers to photographing of the corneal endothelium. The “analysis” refers to analysis of the image of the photographed corneal endothelium. The photographing and analysis may be performed along with the data retrieval as described below. A case where the data retrieval is selected will be described later (FIG. 7). When the photographing or the photographing and analysis is selected, a standby screen CS is displayed on the monitor 9 as shown in FIG. 11.

Then, the anterior segment observing system 36 of the photographing system 5 is activated and the CCD camera 8 for photographing the condition of the subject is also activated. A message that prompts the user or the like to enter necessary items and guides the user or the like to look into a view port (objective) of the photographing device 5 after entering (S9) and fix his/her eyes on a lighted fixation target lamp 42 is displayed on the standby screen CS as a server message. Or, these messages are issued in the form of a synthesized voice through the speaker 11. The user or the like enters the subject information such as the subject name, the birthday of the subject, the illness history of the subject, to the standby screen CS (S9). Then, the user or the like looks into the objective 45 through the view port and fixes his/her eyes on the fixation target lamp 42 (FIG. 3) (S10). The condition of the subject photographed by the CCD camera 8 and the image of the anterior segment photographed by the anterior segment observing optical system 36 are transmitted to the server machine (Sl). The anterior segment image is also displayed on the standby screen CS.

In the server machine 3, it is judged whether or not the transmitted subject condition matches information indicating an appropriate condition stored in the instruction and advice information storing means (S12). When there is difference between them, the instruction or advice for guiding the user to be in the appropriate condition is transmitted to the terminal 2 (S13). In the terminal 2, this instruction or advice is made through the monitor 9 or the speaker 11 (S14).

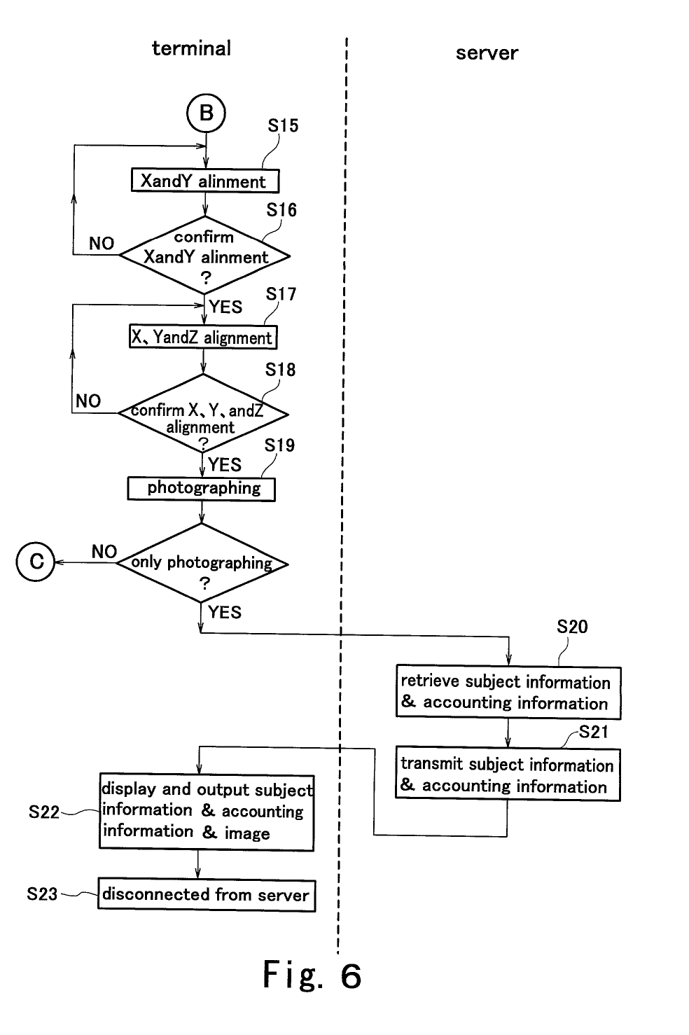

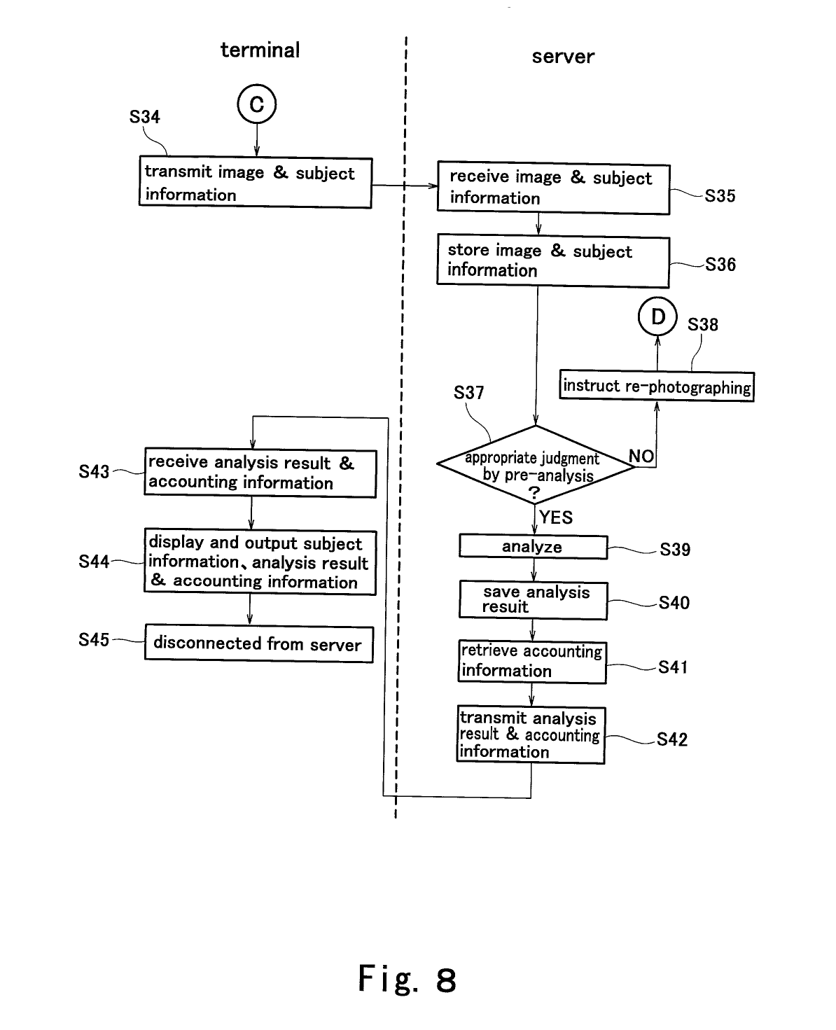

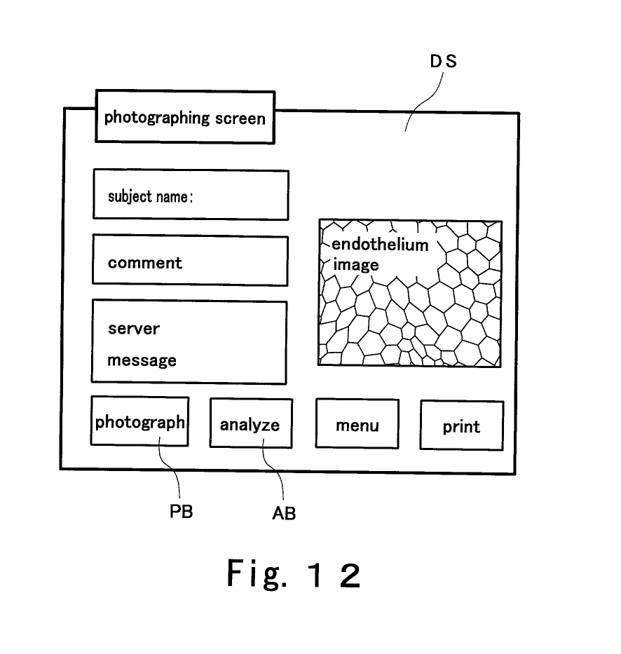

When it is judged that the subject is in the appropriate condition, an instruction for photographing is transmitted to the terminal 2 from the server machine 3 and the photographing operation starts automatically or by the operation of the photographing button PB on the standby screen CS by the user or the like. Specifically, as shown in FIG. 6, the control portion 6 of the photographing device 5 controls the photographing system 5a to perform an alignment operation in X and Y directions (S15, S16). Then, the alignment (focusing) in the Z direction is performed (S17). When the alignment and focusing is confirmed (S18), the photographing control portion 6a controls the xenon tube 37 to emit light. Thus, the corneal endothelium is photographed (S19). The resulting anterior segment image and the corneal endothelium image are stored in the image storing means 16. A photographing screen DS is displayed on the monitor 9 as shown in FIG. 12, and the corneal endothelium image is displayed on the screen DS. A case where the analysis is selected on the menu screen BS will be mentioned later (FIG. 8).

In the server machine 3, automatically, the fee is calculated and the account balance of the user or the like is checked based on the information stored in the first accounting storing means 23 (S20). The accounting information and the subject information are transmitted to the terminal 2 (S21). In the terminal 2, the corneal endothelium image, and the accounting information and the subject information as the server message are displayed on the photographing screen DS (S22). The user or the like can print out these information.

When re-photographing is demanded, the photographing button PB is pressed on the photographing screen DS to cause the standby screen CS to be redisplayed on the monitor 9 and the above-described activation is repeated. If the photographing device 5 comprises a plurality of fixation target lamps (not shown) provided at predetermined positions, on which the user or the like fixes his/her eyes, and selection buttons for selecting the corneal portions to be photographed respectively associated with these fixation target lamps are provided on the photographing screen DS, then the corresponding button is selected in every photographing to allow different portions of the corneal endothelium to be photographed.

In the case where “only photographing” is selected on the menu screen BS, activation is terminated (S23). In addition to this, as shown in FIG. 12, by pressing the analysis button AB on the photographing screen DS at this point, the analysis can be easily selected.

In the case where the data retrieval is selected on the menu screen BS (FIG. 10), the standby screen CS is displayed on the monitor 9 of FIG. 11. As shown in FIG. 7, the user or the like enters the subject information in the same way as described above (S24). When the terminal 2 is provided with a data base in which the subject information or the past analysis result of the subject (including the past image) or the like is stored (S25), the associated subject information or past analysis result is retrieved therefrom (S26).

On the other hand, in the case where such data base is not provided in the terminal 2 or such data base is provided in the terminal 2 but the subject information or the like is not found in such data base, a request for permission to use the server data base is automatically transmitted to the server machine 3 (S27). Receiving the request at the server 3 (S28), retrieval from the data base 22a is conducted and the retrieved information is transmitted to the terminal 2 (S29, S30, S31). At this time, accounting on the terminal 2 is conducted based on the first accounting information storing means 23. In the terminal 2, the subject information, fees and the like are displayed on the monitor 9 (S32). As a display screen, the photographing screen DS or the analysis screen ES may be used. The user or the like can print out this information. At this point, the terminal 2 is disconnected from the server machine 3 (S33).

In the case where the analysis is selected on the menu screen BS (FIG. 10), an operation is performed following the flows of FIGS. 5, 6. When the photographing of FIG. 6 is completed, it goes to the flow of FIG. 8. To be specific, the anterior segment image and the corneal endothelium image are transmitted to the server machine 3 (S34). In the server machine 3, the received (S35) anterior segment image and corneal endothelium image are stored in the analysis result storing means 21 (S36) and the analyzing means prejudges whether or not these images can be analyzed (S37). When judging that these images cannot be analyzed, the analyzing means 20 transmits the instruction for re-photographing to the terminal 2 (S38). On the other hand, when judging that these images can be analyzed, the analyzing means 20 starts analysis and transmits a message stating “the image is being analyzed” to the terminal 2.

The analyzing means 20 geometrically performs image analysis such as smoothing, shading compensation, floating binalization, thinning, and dot noise reducing to extract a line imaging of the cell. Then, the minimum cell area of the corneal endothelium, the maximum cell area of the endothelium, the average cell area of the endothelium, the number of analyzed corneal endothelium cells, the number of corneal endothelium cells per unit area, standard deviation of corneal endothelium cell areas, a variation coefficient of the corneal endothelium cell areas, the rate of appearance of the hexagonal cell, and the like are calculated (S39). The analysis result is stored in the server machine 3 (S40) and transmitted to the terminal 2 (S42). Then, the accounting information is retrieved from the second accounting information storing means 24 and accounting on the terminal 2 is conducted (S42).

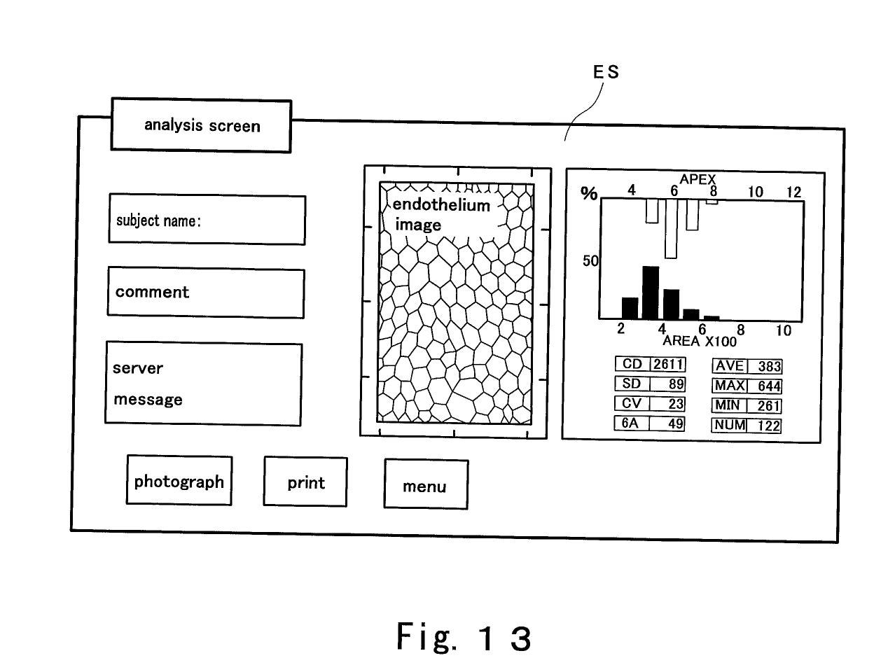

In the terminal 2, the analysis result is received (S43) and an analysis screen ES is displayed on the monitor 9 as shown in FIG. 13. In a bar graph displayed on the analysis screen ES, upper bars indicate distribution (%) representing percentage of how many angles polygon cells have and lower bars indicate distribution of cell areas (%). CD denotes a cell density (the number of cells per 1 square millimeter). SD denotes the standard deviation of the cell areas. CV denotes the variation coefficient (standard deviation/average cell area ×100). 6A denotes the rate (%) of appearance of the hexagon cell. AVE denotes the average cell area (μsquare meter). MAX denotes the maximum cell area (μsquare meter) and MIN denotes the minimum cell area (μsquare meter). NUM denotes the number of analyzed cells. The user or the like can print out the information (including the cell image) displayed on the monitor 9 of FIG. 8 as necessary (S44). At this point, the terminal 2 is disconnected from the server machine 3 (S45).

In the system of this embodiment, a one-to-one connection correspondence may exist between the server machine and the terminal. Alternatively, a plurality of terminals may be connected to one server machine, or to a plurality of server machines. For example, a number of terminals may be connected to a fewer number of server machines.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds thereof are therefore intended to be embodied by the claims.

What is claimed is:

1. A corneal endothelium analysis system comprising:
a terminal; and
a server connected to the terminal through a network, wherein
the terminal comprises means for photographing corneal endothelium and first transmission/receiving means for transmitting an image of the photographed corneal endothelium to the server and receiving an analysis result from the server, and
the server comprises second transmission/receiving means for receiving the image of the corneal endothelium from the terminal and transmitting the analysis result to the terminal, and means for analyzing the image of the corneal endothelium.

2. The corneal endothelium analysis system according to claim 1, wherein the photographing means comprises an anterior segment observing optical system, an alignment optical system, an illumination optical system, a photographing optical system, and a photographing control portion, for automatically photographing the corneal endothelium.

3. The corneal endothelium analysis system according to claim 1, wherein the terminal further comprises first storing means for storing the image of the corneal endothelium, first control means for controlling the first storing means and the first transmission/receiving means, and means for operating the first control means.

4. The corneal endothelium analysis system according to claim 3, wherein the terminal further comprises means for displaying the image of the corneal endothelium and the analysis result under control of the first control means.

5. The corneal endothelium analysis system according to claim 3, wherein the terminal further comprises means for outputting the image of the corneal endothelium and the analysis result under control of the first control means.

6. The corneal endothelium analysis system according to claim 3, wherein the terminal further comprises means for entering subject information and/or information used for obtaining an access right of the server under control of the first control means.

7. The corneal endothelium analysis system according to claim 6, wherein the terminal further comprises second storing means for storing control information enabling the photographing means to perform photographing on condition that permission to use the photographing means is obtained from the server.

8. The corneal endothelium analysis system according to claim 1, wherein the server further comprises second control means and third storing means, the second control means is adapted to control the third storing means, the second transmission/receiving means, and the analyzing means, and the third storing means is adapted to store at least one analysis result selected from a group comprising a minimum cell area of the corneal endothelium, a maximum cell area of the corneal endothelium, an average cell area of the corneal endothelium, the number of analyzed corneal endothelium cells, the number of corneal endothelium cells per unit area, standard deviation of corneal endothelium cell areas, a variation coefficient of the corneal endothelium cell areas and a rate of appearance of a hexagonal cell, and the image of the corneal endothelium.

9. The corneal endothelium analysis system according to claim 1, wherein the terminal further comprises means for observing condition of a subject, and is adapted to transmit information of the condition of the subject observed by the observing means to the server, and the server further comprises sixth storing means for storing information with which it is judged whether or not the subject can be photographed depending on the condition of the subject.

10. The corneal endothelium analysis system according to claim 9, wherein the server is adapted to compare received information of the condition of the subject to the information stored in the sixth storing means and transmit an instruction pertaining to the condition of the subject to the terminal, and the terminal further comprises means for giving the instruction received from the server.

11. The corneal endothelium analysis system according to claim 1, wherein the server further comprises fourth storing means for storing control information used for accounting on the terminal for the use of the photographing means.

12. The corneal endothelium analysis system according to claim 1, wherein the server further comprises fifth storing means for storing control information used for accounting on the terminal for transmission of the analysis result to the terminal.

13. The corneal endothelium analysis system according to claim 1, wherein the server further comprises seventh storing means for storing control information used for transmitting information pertaining to expendable part replacement timing to the terminal according to how the terminal is used.

14. The corneal endothelium analysis system according to claim 13, wherein the photographing means comprises a photographing light source and a detection light source as expendable parts.
